(19) United States
(12) Patent Application Publication
(10) Pub. No.: US 2024/0327102 A1
(43) Pub. Date: Oct. 3, 2024

(54) FOAM DELIVERY SYSTEM FOR MEDICAL APPLICATIONS

(71) Applicant: AGILEWORKS LLC, Alexandria, VA (US)

(72) Inventor: Jonathan HAASE, Alexandria, VA (US)

(21) Appl. No.: 18/598,350

(22) Filed: Mar. 7, 2024

Related U.S. Application Data
(60) Provisional application No. 63/455,929, filed on Mar. 30, 2023.

(51) Int. Cl. B65D 83/30; A61M 5/31

(52) U.S. Cl. CPC B65D 83/303; A61M 5/31

(57) ABSTRACT

Systems, methods, and devices for delivering foam are provided. A foam delivery system includes a pressurized container configured to store foam material, a control valve configured to selectively dispense the foam material from the pressurized container, and a lumen configured to direct foam from the pressurized container to an application site within a cavity. The lumen can be a dual lumen. The foam delivery system can be coupled with an endoscopy system, a laparoscopy system, or a system used in veterinary applications.

### CROSS-REFERENCE TO RELATED APPLICATION

This application claims benefit of priority of U.S. Provisional Patent Application Ser. No. 63/455,929 filed on Mar. 30, 2023, the entire disclosure of which is incorporated herein by reference.

### TECHNICAL FIELD

The present invention generally relates to a foam delivery system for medical applications, and, in particular, a foam delivery system configured to apply therapeutic agents, antimicrobial agents, anesthetic agents, dyes, or markers to internal body cavities during surgical and endoscopic procedures using various foams that are both persistent and non-persistent.

### BACKGROUND

In medical procedures, particularly minimally invasive surgeries, therapeutic agents, anesthetics, or contrast materials are often applied directly to internal body cavities. However, current methods for using such agents can be imprecise, leading to inadequate coverage, inefficiency, or patient discomfort. The present invention addresses these limitations by providing a foam delivery system that ensures uniform distribution and controlled application of the agents.

### SUMMARY

The invention includes a foam delivery system for medical applications, comprising a reservoir containing a foam-agent mixture, a delivery system, and a control mechanism. In some embodiments, the foam delivery system further includes an electronic control system. The foam delivery system is configured for insertion into a target body cavity, allowing the foam-agent mixture to be applied evenly and effectively. In addition, the foam dissolves after a predetermined period, enabling clear visibility.

The delivery system increases accuracy of the application of the foam-agent to a surface within the cavity which reduces the amount of foam-agent necessary as well as increases the efficacy of foam-agent to reduce sensitivity impact of medical procedure to a patient.

### BRIEF DESCRIPTION OF THE DRAWINGS

The accompanying drawings, which are incorporated herein and constitute a part of this specification, illustrate exemplary embodiments. Together with the general description given above and the detailed description given below, serve to explain the features of the various embodiments.

FIG. 1 is a top view illustrating a foam delivery device according to an embodiment of the present disclosure.

FIG. 2 is a side view illustrating the foam delivery device according to an embodiment of the present disclosure.

FIG. 3 illustrates an alternate view of the foam delivery device according to an embodiment of the present disclosure.

FIG. 4 illustrates a lumen quick connect and disconnect system for a foam delivery device according to an embodiment of the present disclosure.

FIG. 5 illustrates a foam delivery device with a dual-lumen according to an embodiment of the present disclosure.

FIG. 6 illustrates a foam delivery device with a syringe according to an embodiment of the present disclosure.

FIG. 7 illustrates a foam delivery device with a pump according to an embodiment of the present disclosure.

FIG. 8 illustrates a foam delivery device with a control panel according to an embodiment of the present disclosure.

### DETAILED DESCRIPTION

The various embodiments are described in detail with reference to the accompanying drawings. Whenever possible, the same reference numbers are used throughout the drawings to refer to the same or like parts. References made to particular examples, details, and representative materials, methods, and implementations are for illustrative purposes only, and thus do not, and are not intended to, limit the scope of the various embodiments of the claims.

The following description with reference to the accompanying figures is provided to assist in a comprehensive understanding of various embodiments of the present disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used to enable a clear and consistent understanding of the present disclosure. Accordingly, it should be apparent to those skilled in the art that the following description of various embodiments of the present disclosure is provided for illustration purposes only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

The terms “have”, “may have”, “can have,” “include”, “may include”, “can include”, “comprise”, and the like used herein indicate the existence of a corresponding feature (e.g., a number, a function, an operation, or an element) and do not exclude the existence of an additional feature.

The terms “A or B”, “at least one of A and/or B”, or “one or more of A and/or B” may include all possible combinations of items listed together. For example, the terms “A or B”, “at least one of A and B”, or “at least one of A or B” may indicate all the cases of (1) including at least one A, (2) including at least one B, and (3) including at least one A and at least one B.

The terms “first”, “second”, and the like used herein may modify various elements regardless of the order and/or priority thereof, and are used only for distinguishing one element from another element, without limiting the elements. For example, “a first element” and “a second element” may indicate different elements regardless of the order or priority. For example, without departing the scope of the present disclosure, a first element may be referred to as a second element and vice versa.

It will be understood that when a certain element (e.g., a first element) is referred to as being “operatively or communicatively coupled with/to” or “connected to” another element (e.g., a second element), the certain element may be coupled to the other element directly or via another element (e.g., a third element). However, when a certain element (e.g., a first element) is referred to as being “directly coupled” or “directly connected” to another element (e.g., a second element), there may be no intervening element (e.g., a third element) between the element and the other element.

The term “configured (or set) to” as used herein may be interchangeably used with the terms, for example, “suitable for”, “having the capacity to”, “designed to”, “adapted to”, “made to”, or “capable of”. The term “configured (or set) to” may not necessarily have the meaning of “specifically designed to”. In some cases, the term “device configured to” may indicate that the device “may perform” together with other devices or components. For example, the term “processor configured (or set) to perform A, B, and C” may represent a dedicated processor (e.g., an embedded processor) for performing a corresponding operation or a general-purpose processor (e.g., a central processing unit (CPU) or an application processor) for executing at least one software program stored in a memory device to perform a corresponding operation.

The terminology herein is only used for describing specific embodiments and is not intended to limit the scope of other embodiments. The terms of a singular form may include plural forms unless otherwise specified. The terms used herein, including technical or scientific terms, have the same meanings as understood by those of ordinary skill in the art. Terms defined in general dictionaries, among the terms used herein, may be interpreted as having meanings that are the same as, or similar to, contextual meanings defined in the related art, and should not be interpreted in an idealized or overly formal sense unless otherwise defined explicitly. Depending on the case, even the terms defined herein should not be such interpreted as to exclude various embodiments of the present disclosure.

The word “exemplary” is used herein to mean “serving as an example, instance, or illustration.” Any implementation described herein as “exemplary” is not necessarily to be construed as preferred or advantageous over other implementations.

The systems and methods of foam delivery for medical applications in accordance with the invention allow for applying a foam agent mixture within a target body cavity. The foam agent can be delivered and applied within the body cavity with increased accuracy before, during, or after a medical procedure or application. After the foam is applied to the cavity, the foam may dissolve after a predetermined time period enabling the medical procedure to proceed without significant foam impacting visibility within the cavity. The accuracy of application of the foam agent to a surface within the cavity can be increased in accordance with the embodiments of the invention which can reduce an amount of foam agent introduced to the cavity as well as the efficacy of the foam agent to reduce impact of medical procedure to a patient.

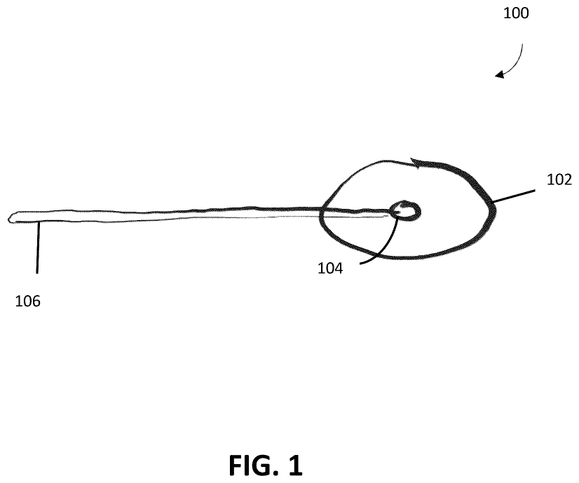

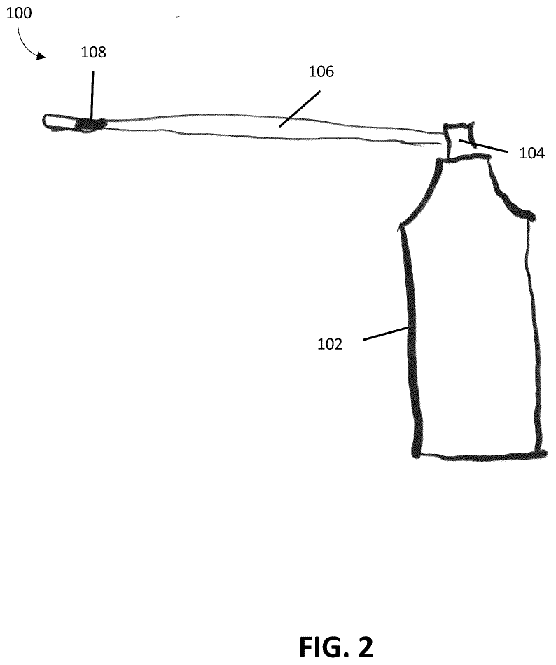

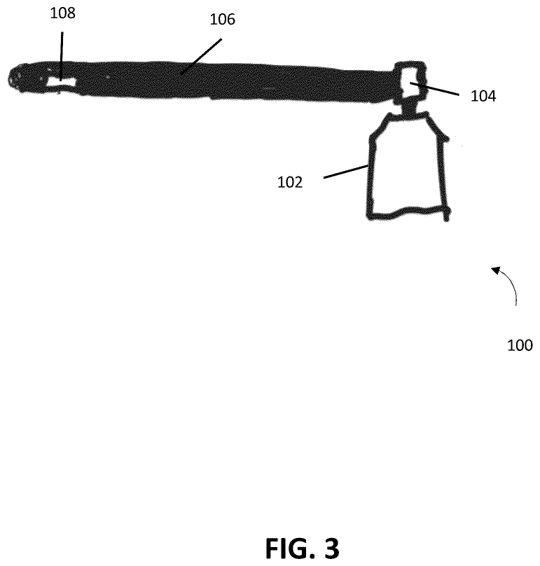

FIG. 1 illustrates a top view of one embodiment of the illustrating a foam delivery device according to an embodiment of the present disclosure. FIG. 2 illustrates a side view of the foam delivery device according to an embodiment of the present disclosure. FIG. 3 illustrates another side view of the foam delivery device according to an embodiment of the present disclosure.

Referring to FIGS. 1-3, foam delivery device 100 can include a reservoir 102, a control valve 104, and a lumen 106.

In an exemplary embodiment, reservoir 102 can be a pressurized container such as a canister. Reservoir 102 can be configured to enclose a foam agent material and propellant within the pressurized container. Reservoir 102 can also be configured to withstand the pressure of propellent gas stored within reservoir 102. In an exemplary embodiment, reservoir 102 can be pressurized by standard aerosol pressurization or the control valve 104.

In an exemplary embodiment, reservoir 102 can be configured with a specific capacity for holding the foam agent mixture. The size, shape, and/or capacity of reservoir 102 can be selected based on the foam agent and/or propellant as well as the intended application of the resulting foam. The dimensions of reservoir 102 can include a range of capacities, from a small capacity of a few milliliters for minor procedures to larger capacities for more extensive procedures.

Reservoir 102 can be made of any kind of material that can withstand the pressure of converting the foam agent into a foam while providing structure for enclosing the foaming agent and propellant, particularly within a medical environment. In some embodiments, reservoir 102 can be made from medical-grade materials like stainless steel or high-quality plastics. However, any kind of material that can withstand the pressure while providing structure for storing the internal components may be used.

The foam agent mixture can be a combination of ingredients specifically formulated to produce foam. In an exemplary embodiment, the foam agent mixture can include a surfactant or foaming agent to help stabilize bubble formation. As the foam agent mixture is released from reservoir 102, the foam agent mixture can undergo a process such that gas from the propellant interacts with the foam agent mixture to create foam. The foam agent mixture can be customized to include therapeutic agents, anesthetic agents, dyes, or fluorescent markers, depending on the medical application. The foam agent may be premixed before the canister is loaded into reservoir 102 of the foam delivery device 100. Alternatively, the foam agent may mix after loading within reservoir 102 but before application of the foam into the cavity during a medical procedure. When the propellant and the foam agent are maintained within separate chambers, a chamber associated with the propellant can be provided between the chamber associated with the foam agent and the control valve 104. Reservoir 102 can be sealed to maintain pressure within reservoir 102 as well as prevent leakage of the foam agent or propellant gas from reservoir 102. The seal can be configured to maintain the pressure within reservoir 102 while also preventing any air in the external environment from penetrating within reservoir 102. The material used to seal reservoir can be any material configured to provide a secure and airtight closure to prevent leaks and maintain the structural integrity of reservoir 102. For example, seal material can include rubber, elastomer, plastic, metal, composite materials, Polytetrafluoroethylene (PTFE), or the like.

Control valve 104 can be any valve capable of facilitating the release of foam to an application site. That is, control valve 104 can be configured to activate the migration of foam agent from within a chamber of reservoir 102 as well as exposure to the propellant within reservoir 102 to expel foam from reservoir 102 to an application site. Control valve 104 can include a stem that extends within reservoir 102. In an exemplary embodiment, the stem of control valve 104 can connect with a sealing mechanism to control the start and stop of the foam flow. Control valve 104 can be configured such that pressure is applied via various pumps or control systems including an electronic pump, a control system, or a manual pump.

Lumen 106 can be a passage, channel, tube, or pathway configured to guide the foam from reservoir 102 to a foam application site. Lumen 106 can be a single lumen or a dual-lumen. In FIG. 1, a single lumen is illustrated.

Lumen 106 can be made of any type of material including medical grade materials such as plastics, stainless steel, or silicone. Referring to FIGS. 2 and 3, lumen 106 can further include port 108 formed in a side of lumen 106. While port 108 is illustrated in FIG. 2 as being towards the tip closer to the outlet hole formed at the end of lumen 106, port 108 can be provided at any location along lumen 106.

In an exemplary embodiment, foam can be expelled from lumen 106 through port 108 onto the foam application site. In an additional or alternative embodiment, foam can be expelled from an outlet hole formed at the end of lumen 106. That is, foam may be selectively output from lumen 106 through an outlet hole formed at the end of lumen 106, through port 108, or through a combination thereof. While one port 108 is illustrated in FIG. 2, a plurality of ports can be provided at various locations along the length of lumen 106.

In an exemplary embodiment, a pressure gauge can be in communication with reservoir 102, the propellant chamber, and/or lumen 106. The pressure gauge can be configured to monitor the pressure inside reservoir 102 to ensure that pressure within reservoir 102 maintains adequate operating pressure or monitor the pressure of the foam expelled from reservoir 102. Additionally or alternatively, the pressure gauge can monitor pressure within lumen 106 before, during, and/or after foam application.

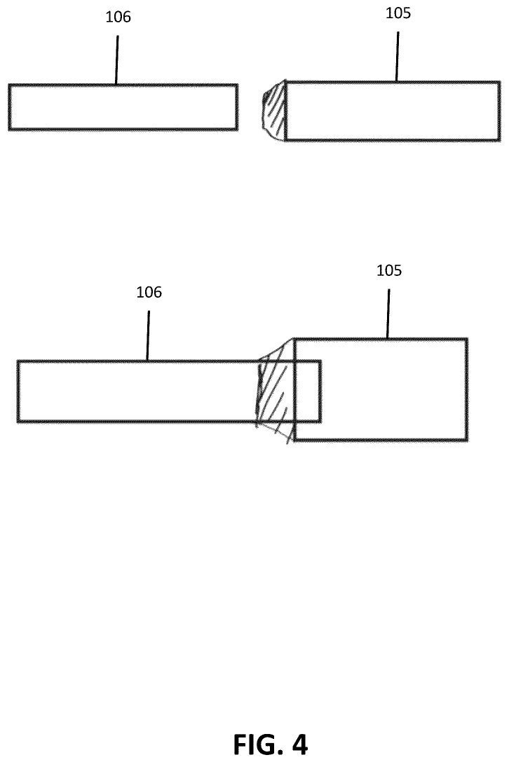

FIG. 4 illustrates a lumen quick connect and disconnect system for a foam delivery device according to an embodiment of the present disclosure.

Referring to FIG. 4, lumen 106 can be configured to be removably coupled with reservoir 102 via a connector 105 in communication with control valve 104.

Connector 105 can be any type of connector or fitting configured to facilitate connection and disconnection between lumen 106 and control valve 104. For example, connector 105 can be a slip fitting or a fitting that includes threading to allow lumen 106 to be threaded onto connector 105.

Lumen 106 can include a flexible material that can be reused or disposable. In an exemplary embodiment, lumen 106 can be stored, assembled, or shipped quickly and easily while minimizing the space required for storage of lumen 106. Lumen 106 is configured to be removably coupled from the elements of the foam delivery device 100 such that the system can be easier to use, and transport and lumen 106 can be stored or disposed of separately from the reservoir 102. In an exemplary embodiment, lumen 106 can be disposed of quickly and easily after a procedure is performed.

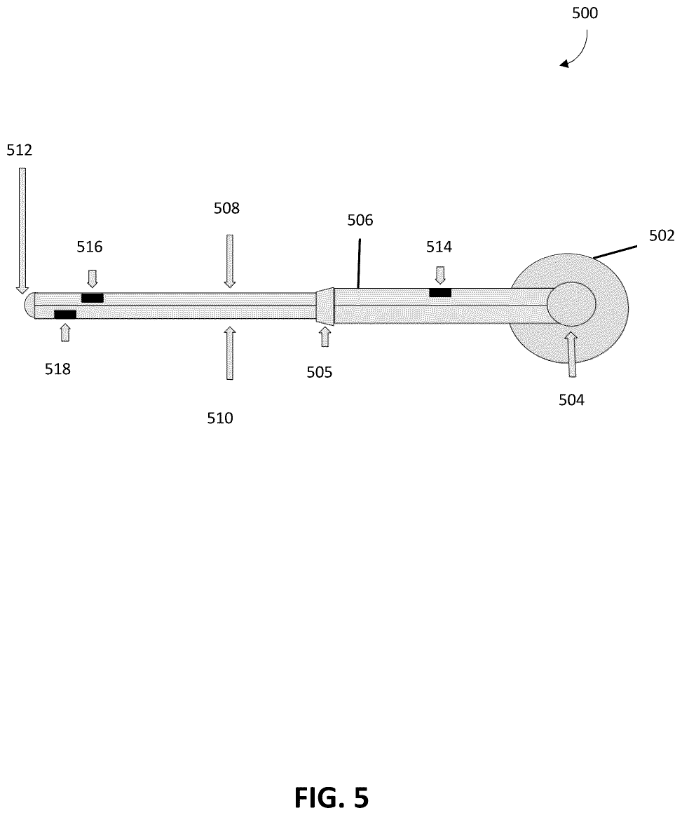

FIG. 5 illustrates a foam delivery device with a dual-lumen according to an embodiment of the present disclosure.

Referring to FIG. 5, a foam delivery device 500 can include a reservoir 502, a control valve 504, and a dual lumen 506.

Reservoir 502 can be similar to reservoir 102 and control valve 504 can be similar to control valve 104.

Dual lumen 506 can include a plurality of passages, channels, tubes, or pathways configured to guide the foam from reservoir 502 to a foam application site. In an exemplary embodiment, the passages, channels, tubes, or pathways can be distinct from each other such that the foam travels through each passage, channel, tube, or pathway from reservoir 502 to the application site. Alternatively, dual lumen 502 can have different configurations for different portions of the dual lumen 506. For example, a single passage, channel, tube, or pathway can be coupled with control valve 504 and two lumens can couple at connector 505 such that the foam is directed between the two lumens to the application site. In an exemplary embodiment, dual lumen 506 can include a termination cap 512 that seals one or both of the passageways of dual lumen 506. Termination cap 512 can be shaped and configured to reduce harm or discomfort during insertion or removal of dual lumen506 from the cavity.

One or more ports (514, 516, 518) can be formed in dual lumen 506. While three ports are illustrated in FIG. 5, dual lumen 506 can include any number of ports in various locations. For example, ports 516 and 518 can be provided at opposite sides and at different locations in dual lumen 506. However, placement of ports 514, 516, and 518 can be provided in different locations and arrangements than that illustrated in FIG. 5. In an exemplary embodiment, the configuration and dimensions of dual lumen 506 can be customized and configured based on a specific target cavity and application requirements.

In an exemplary embodiment, ports 516 and 518 can be configured such that foam passes through ports 516 or 518 to an application site. Port 514 can be configured as an overflow port. That is, dual lumen 506 may be provided into a cavity and foam may be delivered to the application site(s) in response to applying pressure to control valve 504. Ports 516 and 518 can be provided on opposite sides of dual lumen 506 and foam can be provided to the application site(s). When the cavity is filled and/or an excess of foam is deposited in the cavity associated with the application site, the excess foam can be directed from the cavity to outside the body through port 514. Port 514 can be configured to allow foam to flow back into dual lumen 506 from the cavity.

While not illustrated in FIG. 5, a suction or vacuum device can be included or coupled with reservoir 502 to aid in removing foam from the cavity.

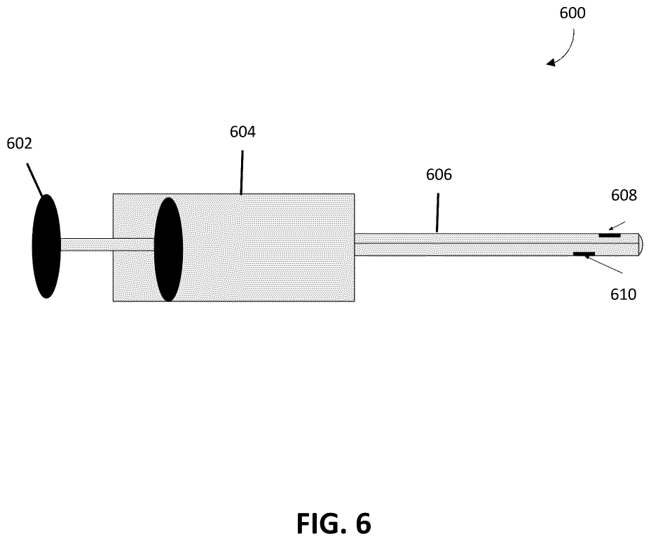

FIG. 6 illustrates a foam delivery device with a syringe according to an embodiment of the present disclosure.

Referring to FIG. 6, foam delivery syringe device 600 can include plunger 602, barrel 604, and dual-lumen 606.

Plunger 602 can be configured to create a seal within the barrel and control the flow of foam into and out of barrel 604. For example, when plunger is pulled back, a vacuum is created that allows foam to be drawn up into barrel 604. When plunger is pushed down, foam can be distributed to the application site via the dual-lumen 606.

Dual lumen 606 can be configured to direct foam from barrel 604 to the application site in a cavity. While dual-lumen 606 is illustrated in FIG. 6, a single lumen could alternatively be used. In an exemplary embodiment, foam can be provided to the application site via port 608 of dual-lumen 606 and any excess foam can be removed from the cavity via port 610.

In an exemplary embodiment, barrel 604 of foam delivery syringe device 600 can be filled with an agent and a foaming agent. Dual-lumen 606 can be inserted into target cavity and plunger 602 can be depressed once the dual-lumen 606 is provided in the cavity. Barrel 604 can be translucent to allow for visual observation of foam application. The application procedure can be complete upon all foam being dispensed from barrel 604 or a visual observation of overflow from the application cavity may be observed. In a case that excess foam is dispensed, plunger 602 can be pulled back creating a vacuum which would allow foam to return to dual-lumen 606 via port 610.

In an exemplary embodiment, foam delivery syringe device 600 can be prefabricated and/or preloaded with foam and foam agent premixed inside the barrel of the syringe. The size of barrel 604 can vary based on foam and/or application and can include various sizes and plunger dimensions for operations. Foam delivery syringe device 600 allows for more precise application to smaller cavities than a larger foam delivery system.

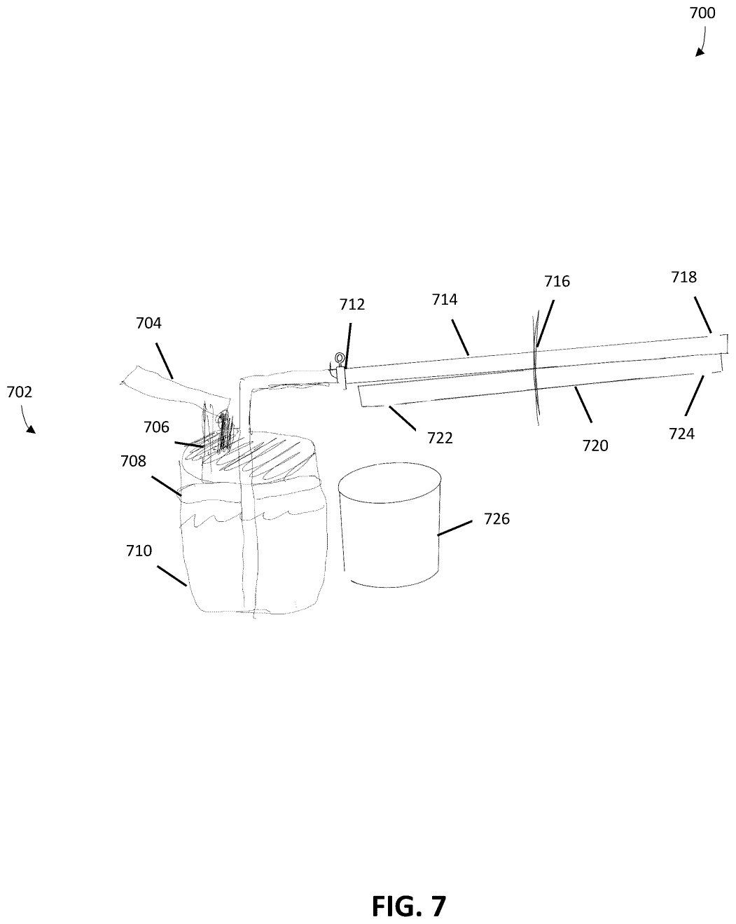

FIG. 7 illustrates a foam delivery device with a pump according to an embodiment of the present disclosure.

Referring to FIG. 7, foam delivery pump system 700 can include pump assembly 702, valve 712, delivery lumen 714, return lumen 720, and overflow catchment 726.

Pump assembly 702 can include lever 704, fulcrum 706, piston/plunger 708, and barrel assembly 710. Lever 704 can be configured to receive a mechanical force. Lever 704 can rotate around fulcrum 706 such that the force applied to lever 704 is transferred to piston 708 via fulcrum 706. Piston or plunger 708 can create a seal and provide structure to create a vacuum within barrel assembly 710 to pressurize the foam. As the piston moves, it creates a vacuum which allows foam within barrel assembly 710 to be displaced to delivery lumen 714 for delivery into cavity to an application site. After valve 712 is opened, foam can flow into a cavity through port 718 of delivery lumen 714. Any overflow from the cavity can migrate to the overflow catchment 726 via ports 722 and 724 in the return lumen 720.

In an exemplary embodiment, the pump can include a lever and fulcrum attached to a plunger to generate pressure. The piston can be connected to a barrel with a foam and agent mixture. When the lever is pressed, pressure can be generated in the barrel of the reservoir. When the control mechanism is opened, the pressure from the operator can allow the foam and agent to flow through the lumen into the targeted cavity.

The lever, fulcrum, and barrel assembly can come in various sizes. The smallest size can include a lever that is 15 cm long and attached to a 7 cm barrel. The size range of the barrel can be 152 cm in diameter and up to 152 cm in height.

Valve 712 can be any valve configured to prevent a flow of foam until a threshold level of pressure is created within barrel assembly 710 such as a gate valve, ball valve, disc valve, etc. In an exemplary embodiment, as illustrated in FIG. 7, valve 712 can be a manual turn valve that is configured to be operated by manually turning the handle.

Delivery lumen 714 and return lumen 720 can include a quick disconnect 716 to allow delivery lumen 714 and return lumen 720 to be removably attached to pump assembly 702. The size of delivery lumen 714 and/or return lumen 720 can vary with application and/or foam agent materials. In an exemplary embodiment, a length of delivery lumen 714 and/or return lumen 720 can be in the range from 1 cm to 170 cm in length and a diameter of 2 mm to 50 mm. However, any length, diameter, or shape of the delivery lumen 714 and/or return lumen 720 may be used based on the application.

Overflow catchment 726 can be configured to catch any overflow foam and prevent spillage to preserve a hygienic and clean medical space.

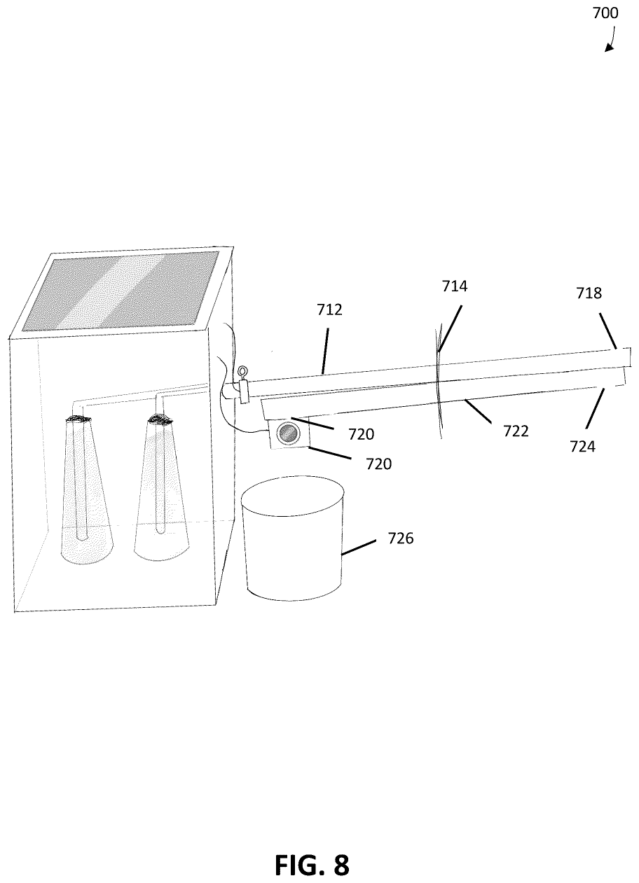

FIG. 8 illustrates a foam delivery device with a control panel according to an embodiment of the present disclosure.

Referring to FIG. 8, electronically controlled foam delivery pump system 800 can include pump assembly 802, control panel 804, valve 806, delivery lumen 808, return lumen 812, overflow detection sensor 818, and overflow catchment 820.

Pump assembly 802 can include premixed, pressurized units filled with foam. In an exemplary embodiment, an electronic control interface can control the pressurization and delivery of foam from pump assembly 802 to the application site via control panel 804.

In an exemplary embodiment, after receiving a request to initiate foam application, control panel 804 can monitor and pressurize the units filled with foam. After the units reach a predetermined threshold, the control panel 804 can send a signal to actuate valve 806 such that foam enters delivery lumen 808 and is delivered in a cavity to an application site via port 808. Foam may enter port 814 of return lumen 812 and exit return lumen 812 via port 816. In an exemplary embodiment, a quick disconnect 810 can be included in electronically controlled foam delivery pump system 800.

Valve 806 can be electronically or manually controlled to allow for precise regulation of delivery of the foam mixture. This ensures accurate delivery of the desired foam volume to the target cavity. In an exemplary embodiment, valve 806 can be an electronic valve connected to the control system. The control system can translate user commands into open and closed commands to articulate the valve and regulate the flow of the foam.

Overflow detection sensor 818 can monitor foam flowing out of return lumen 812 into overflow catchment 820. In an exemplary embodiment, overflow detection sensor 818 can be a camera or any other sensor capable of detecting the foam (light, motion, etc.). Overflow detection sensor 818 can monitor the flow of the foam into the target cavity. In an exemplary embodiment, overflow can be monitored by a camera that visually detects the overflow and automatically sends a control signal to shut off the foam flow when a predetermined threshold is met. In a semi-automatic mode, an operator can visually monitor the overflow and stop the foam flow manually when overflow is detected.

Control panel 804 can be a digital interface configured to allow a user to configure and customize variables associated with the electronically controlled foam delivery pump system 800 such as a duration of foam delivery, an amount of foam provided during delivery, and a rate of foam delivery to cavity. Control panel 804 can include at least one of one or more processors, a display, a memory, or a communication interface. Usage data can be stored in memory. For example, data can be stored for quality assurance, documentation, and research purposes. In an exemplary embodiment, data stored in the memory can be wirelessly transmitted to a remote server or database for further analysis.

In an exemplary embodiment, the control system can be connected to foam reservoir(s) of pump assembly 802 and the delivery lumen 808. A delivery reservoir can be filled with the desired foam and agent mixture. Delivery lumen 808 can be inserted into the target body cavity. A flow of the foam can be started by activating the control mechanism. While the foam is flowing, the overflow detection sensor 818 can monitor any output from the return lumen 812 to ensure that the foam adequately fills the cavity and prevents any overfilling. The flow of foam can be stopped by deactivating the control mechanism when a predetermined amount of foam has been delivered or when the overflow detection mechanism indicates the cavity is full. The delivery lumen 808 can be carefully removed from the body cavity.

Any of the above foam delivery systems can be utilized in various medical applications including endoscopy, laparoscopy, and veterinary applications. For example, the foam delivery system can be adapted for seamless integration with existing endoscopic or laparoscopic systems, allowing for uniform distribution of foam agents during minimally invasive procedures. In another example, the foam delivery system can be used for veterinary medicine with customizable configurations and settings to cater to various animal species and procedural requirements.

Various foams or foaming agents can be any medicament chosen from any physiologically or pharmacologically active substance that produces a local or systemic effect when released in a biological environment. The active medicament can be inorganic or organic compounds including drugs that act on the nervous system, drugs that act on tissues, muscles, and organs, analgesics, anti-inflammatory agents, prostaglandins, antimicrobials, anti-virals, antifungal agents, and hormones. Specific drugs and doses may be found in REMINGTON'S PHARMACEUTICAL SCIENCES, 14th Edition, 1970; Mack Publishing Co., Easton, PA.; and Goodman and Gilman, THE PHARMACOLOGICAL BASIS OF THERAPEUTICS, 7th Edition, 1985; MacMillian Company, New York, New York; both of which are incorporated herein by reference.

The foam delivery system can use persistent foams which are injected via the delivery and control systems described above. The foam can be left in the target cavity to apply various agents, such as a combination of antimicrobial and therapeutic agents following surgery in the uterus to aid in healing and prevent infection. The foam can be later removed by a health care professional after application time is complete. In an exemplary embodiment, foam can be applied to fill a uterus after surgery is performed. Target agents included in the foam can assist in recovery, such as a mixture of antimicrobial and therapeutic agents. After a day, the foam can be removed ensuring the agents are in contact with the uterus for a day.

In an alternative embodiment, the foam can be transient such that it dissolves after a period of time of being in contact with the cavity. For example, the foam can be deposited and after application, it can expand and fill the cavity. The intended agents in the foam then dissolve with no further action by medical professionals which saves time and effort. In an exemplary embodiment, after a procedure on the uterus, a dissolving foam mixed with target agents can be applied to the uterus. The foam can start to dissolve after the target agent has made contact with the uterus.

While certain cavities are identified above, any of the foam delivery systems described above can be configured to be used with the intended cavities. For example, for small cavities, the agent reservoir, delivery device, pressure generating system, and control system can be smaller and shorter. For larger cavities, the agent reservoir, the agent reservoir, delivery device, pressure system, and control system can be larger and longer.

The foam can include agents that facilitate tissue repair, healing, regeneration, promote faster recovery, and improve patient outcomes following a medical procedure. The foam delivery systems can be designed for compatibility with endoscopic devices, facilitating the integration of foam delivery system into advanced medical procedures or technologies.

The foam, an agent, an agent reservoir, a delivery system, a pressure generating system, and a foam flow control system can be modular, allowing for easy replacement, maintenance, or upgrading of individual components, as well as customization to suit the needs of specific medical procedures or patient populations.

For example, a small delivery system can be connected to a large reservoir if a patient has small openings in a specific cavity. This allows the foam to be delivered to many cavities regardless of the patient's anatomy.

The foam delivery system can further include safety features, including a control system that only releases foam from the reservoir when the control assembly is opened. A return lumen can be configured to allow excess foam to return from the cavity to the overflow lumen port, which vents to the atmosphere. In an alternative embodiment, a vacuum device can be used to create a negative pressure environment within the cavity which causes the foam to be drawn out of the cavity. In some embodiments, the overflow detection system can trigger an automatic shutoff function corresponding to the flow of foam.

The foam delivery system can minimize the risk of cross-contamination or infection between patients, contributing to improved patient safety and infection control practices in medical settings.

As described above, the lumen system can be detachable via a detachment system such that the lumen are disposable. Alternatively, the lumen system can be detachable via a detachment system to be sterilized by standard sterilization techniques.

The foam delivery system can be manual, automated, or semi-automated to reduce inaccuracies during foam mixture application and enhancing the precision of the medical procedure. The automated system can be configured to apply foam using a dual-lumen configuration such that one lumen deposits the foam and the second lumen is used for foam return from the cavity. In an exemplary embodiment, the deposition and return lumen can be separated by 2 mm to 20 cm depending on the cavity in which the foam is applied. Larger cavities can have a greater distance between the two lumens, and smaller cavities can have a smaller distance between the two lumens.

An automated system can include an electronic system with a user interface, an electronically controlled valve to initiate the start and stop of the flow of foam, and a visual foam sensing camera that monitors overflow exit of the foam from the return lumen.

The control system can include non-transitory, computer readable media that is configured to store data. Algorithms configured to deliver, detect, and monitor foam flow and depositing can be stored in the control system. When foam is detected, the algorithm can send an alert signal allowing the system to be aware when the foam exits the overflow lumen indicating the cavity is filled with foam.

In another exemplary embodiment, an operator can turn on the system and select a fill command from the electronic user interface. In response, the system can send a command to the control mechanism opening the foam flow to the tube. A viewport into the delivery tube can monitor the foam flow to the target cavity. A visual or audible signal can alert the operator if no foam flow is detected. If the flow is detected, the foam can continue to flow to the cavity. When the cavity is full of foam, the pressure can exert a force on the foam allowing it to flow through the return lumen to the overflow window. The overflow window can be monitored by a fixed camera that detects foam overflow and sends an automatic shutoff signal to the control mechanism. When the control mechanism is closed, the control system interface records the amount of foam that has flowed into the cavity as well as the time of day and fluid flow into the cavity.

If no overflow is detected after a predetermined time, a message can indicate that no overflow is detected. At the same time, a shutoff signal can be sent to the control mechanism to ensure that excessive foam is not delivered to the cavity. Combining automatic start, monitoring of foam flow, overflow detection, automatic shut off, and no overflow detection combined to make an automated foam delivery system.

In an exemplary embodiment, an operator can first insert the lumen into the target cavity. Once the lumen is inserted, the operator can select the start button on the digital interface. The control mechanism can be opened when the operator has pressed the start button causing foam to flow to the target cavity. The operator can receive a confirmation message that foam is flowing to the target cavity. Once overflow is detected, the system can automatically shut off and record the data for that session. At the same time, it can display a visual signal and/or provide an audible signal alerting the operator of the completion of cavity filling. The pressure required to return the foam to the overflow ensures the cavity is full of foam and provides evidence that can be recorded by the automated system and stored for future reference.

In a semi-automated system embodiment, the elements can be the same, but the software and digital interface can be further used to prompt the technician to verify the foam is flowing to the cavity and any overflow situations. The technician can visually see any overflow and in response press a button on the digital interface shutting off the control system. The semi-automated feature can allow the technician to manually cease the foam flow to the target cavity after verifying that the foam is exiting the overflow.

In a manual system embodiment, the technician can manually open the control valve, visually monitor for overflow, and manually close the control valve when overflow is detected. The technician can have several different ways to start and stop the foam flow. For example, the technician can open the valve to begin the foam flow manually. The technician can monitor the foam flow, and when the foam is detected from the overflow lumen, the technician can manually close the valve stopping foam from flowing to the target cavity.

In another exemplary embodiment, the technician can press a start button on a digital interface connected with the control valve. When the technician presses the start button, fluid can flow as the control valve is opened via an automatic signal and an electronic control valve. The technician can observe the overflow lumen and when overflow is detected, depress the stop button on the control interface. When the control interface stop button is pressed, an electronic signal is sent to the control valve stopping the foam flow.

In another exemplary embodiment, the foam delivery system can include a small portable canister with a control valve configured to be depressed, allowing foam to flow through the tube system. This allows the technician to hold a small can in their hand and depress a manual control valve which commences the foam flow to the target cavity. The technician can continue to depress the valve, allowing the foam to flow until the technician observes overflow from the return lumen. The technician can release the valve which causes the foam to cease to flow. The technician can remove the lumen from the cavity to complete the foam application procedure.

In an exemplary embodiment, a larger reservoir that is pre-pressurized with premixed agents and foams can be used. A manual quarter valve can control the flow of foam connected to the reservoir. The control valve can connect to the lumen delivery system to provide the foam to the cavity. When the technician wants to begin the foam flow, they can open the valve and start the foam flow to the cavity. The technician can monitor the overflow lumen and observe for foam to exit the overflow. When the technician observes foam leaving the overflow lumen, they can turn the valve to the closed position which stops the foam flow to the target cavity. After the foam delivery is complete, the lumen can be removed from the cavity.

Reservoirs 102, 502, barrel 604, and/or pump assemblies 702, 802 can be made of any material configured to store foam or agent, withstand the pressure required to flow foam through the lumen delivery system, and do not alter the properties of the foam or agent. The reservoirs can include one or more therapeutic agents, antimicrobial agents, anesthetic agents, dyes, markers, and foam-forming substance. The foam forming substance can include a surfactant, a gelling agent, or other suitable materials that create a foam consistency when combined with the therapeutic or anesthetic agents.

The reservoir can vary in size ranging from small, handheld reservoirs capable of filling small cavities between 3 cm to 21 cm in diameter to 3 cm to 30 cm in height to large in diameter reservoirs capable of holding many applications of foam ranging from 21 cm to 152 cm in diameter and 15 cm to 152 cm in height.

In an exemplary embodiment, the reservoir can be an aerosol can and the agent reservoir can be included within a container that neither alters the foam agent substances nor contributes anything to them while simultaneously being able to withstand the pressure of the aerosol. The foam mixture can be released through a control valve connecting to a tube.

The control mechanisms can be a valve controlled by an electronic control system that collects user commands from a control system and translates those into opened and closed commands to a control valve. The control mechanism can also be a manual valve that an operator can manually move between an open and closed position to regulate the flow of the foam.

In an exemplary pump embodiment, the foam delivery device can include a pump connected to a reservoir that an operator can manually depress to generate pressure. The pressure can build in the reservoir, and when the control mechanism is open, the foam flows through the reservoir into the tube system. The pump can continually operate to ensure adequate pressure until the foam overflow from the return lumen. When overflow is detected, pressure is released, the control mechanism is closed, and the flow of foam is discontinued which completes the procedure. The technician can then remove the lumen from the cavity.

The control valve or the lever of the pump can be operated with a non-dominant hand to apply pressure while the dominant hand operates the valve to ensure dexterity and control over the foam flow. In a manual embodiment, the operator can apply pressure to the lever with the non-dominant hand while simultaneously opening the control mechanism to begin the flow of foam and agent to the targeted body cavity. The operator can continue to depress the lever generating pressure via a piston system inside of the reservoir until the foam and agent are visually detected from the overflow. When overflow is detected, the control mechanism can be closed. In contrast, the pressure can be released from the non-dominant hand on the lever. The foam can be safely applied to target cavities until filled with foam which can be forced back into the return limit and is visually detected, leaving the overflow.

In an exemplary embodiment, the lumens can measure from 1 cm to 170 cm in length and 2 mm to 55 mm in diameter. The lumens can be configured for insertion into the target body cavity and have a distal end to deliver the foam mixture. Duel lumens can include one lumen to deliver the foam and a second lumen to return the foam to the overflow. Two openings or ports can be on opposite sides of the tubes and separated by 2 mm to 20 cm depending on the cavity the foam is being applied to. Larger cavities can have a greater distance between the two lumens, and smaller cavities can have a smaller distance between the two lumens.

In an exemplary embodiment, the pump mechanism can include an agent reservoir connected to a dispensing tube. The pump handle, constructed from medical-grade material, can allow for the controlled release of the foam mixture through the dispensing tube which can also be inserted into the target body cavity.

In an exemplary embodiment, the control mechanism can allow the user to regulate the release and application of the foam mixture. This can be achieved using an adjustable valve. The valve can be controlled in a manual embodiment by turning a visible control lever which clearly shows the open and closed position of the control valve. The valve can be controlled by electrical signals from the control system when an electronic control system is used to start, stop, and monitor foam flow. A digital control panel with a touchscreen interface can set precise release and application settings such as duration, amount, and rate of foam mixture release. This occurs via a control interface where a user can select either fully automatic, semi-automatic, or manual flow process control. The fully automatic configuration can send a signal to the control mechanism that initiates foam flow. The foam can continue to flow until the overflow shows foam returning from the cavity. The overflow can be monitored by a camera that visually detects overflow and automatically sends a control signal to the control mechanism to shut off the control mechanism detecting the foam flow.

In another exemplary embodiment associated with a semi-automated mode, the control system can start foam flow based on a start command which electronically opens the control mechanism and initiates the foam flow. The operator can then monitor the overflow. When the overflow foam is observed, the technician can press a stop button on the control interface, which signals the control mechanism stopping the foam flow.

In an exemplary embodiment, one or more elements of the foam delivery system can be integrated into endoscopic systems. For example, the operator can connect the foam delivery system with integrations and tubing to connect to the endoscopic lumens. Then, the operator could apply foam as discussed above to deliver foam and agents to completely cover a cavity to be used during endoscopic procedures or with endoscopic tools. This can allow medical professionals to use existing endoscopic tools and systems by integrating the foam delivery system to deliver foam for various effects on a particular organ or cavity. The benefit of using the foam delivery system with an endoscopic system is that it quickly fills the volume of the cavity with the foam and the desired agent and then quickly dissolves the foam for the medical professionals to be able to clearly see and operate inside the cavity with the endoscopic system.

In an exemplary embodiment, a control system can also use a digital interface to allow a technician to pre-program several configurable variables, including the amount of foam to flow or the volume of foam to deliver to the cavity so that the foaming procedure is tailorable based on the desires of the technician. This can be done by pre-programming the duration of foam release time into the control mechanism. With the pre-programmed flow times, the control mechanism is open for the desired time the operator configures. The system would have a database of time and volume calculations. To deliver a specific amount of foam based on the user input to deliver the appropriate foam volume. The user can input the volume of foam desired, and the control system can calculate the time the foam is to be delivered to the cavity. Once the foaming procedure commences, the control mechanism can be opened for the pre-programmed amount of time to deliver the correct foam volume to the target cavity. When the time is complete, the control mechanism can be shut, ensuring the correct volume of foam was delivered consistently with the volume of foam delivered variable entered in the control system by the technician before beginning the procedure.

In an exemplary embodiment, the foam delivery system can be used during laparoscopic surgery. The foam delivery system can administer anesthetic agents to the targeted tissue, and reduce pain and discomfort. The foam's consistency can allow for better contact and coverage of the tissue, and its dissolving properties can help maintain visibility for the surgeon.

In an endoscopic procedure, the foam delivery system can be employed to apply dyes or markers, enhancing the visibility of the targeted tissue or organ. The foam's capacity to cover irregular surfaces can aid in ensuring the marker is evenly applied.

In another exemplary embodiment, veterinarians can use the foam delivery system during a surgical procedure on animals to administer therapeutic agents like antibiotics or anti-inflammatory drugs directly to the targeted tissue, providing more effective treatment.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. The specification describes specific examples of accomplishing a more general goal that also may be accomplished in another way. Those skilled in the art will appreciate that the features described above can be combined in various ways to form multiple variations of the invention.

1. A foam delivery system comprising:
a pressurized container configured to store foam material;
a control valve configured to selectively dispense the foam material from the pressurized container; and
a lumen configured to direct foam from the pressurized container to an application site within a cavity.

2. The foam delivery system of claim 1, further comprising:
a connector configured to connect the control valve and the lumen such that the lumen is selectively removable from the connector.

3. The foam delivery system of claim 2, wherein the connector includes a threaded portion.

4. The foam delivery system of claim 1, wherein the lumen comprises one or more ports formed in a side of the lumen.

5. The foam delivery system of claim 4, wherein the lumen is a dual lumen.

6. The foam delivery system of claim 5,
wherein the dual lumen includes a first lumen and a second lumen,
wherein the one or more ports includes a first port and a second port,
wherein the first lumen includes the first port, and
wherein the second lumen includes a second port.

7. The foam delivery system of claim 6,
wherein the foam is delivered to the cavity through the first port in the first lumen, and
wherein excess foam is removed from the cavity through the second port in the second lumen.

8. The foam delivery system of claim 6,
wherein the second lumen further includes a third port, and
wherein the foam is delivered to the cavity through the third port.

9. The foam delivery system of claim 1,
wherein the foam delivery system is a syringe device,
wherein the pressurized container is a barrel, and
wherein the control valve is a plunger.

10. The foam delivery system of claim 1,
wherein the foam delivery system is a pump system,
wherein the pressurized container is a pump assembly, and
wherein the control valve is a valve provided on the lumen.

11. The foam delivery system of claim 10,
wherein the lumen is a dual lumen including a first lumen and a second lumen,
wherein the first lumen includes a first port through which foam is delivered to the cavity,
wherein the second lumen includes a second port and a third port,
wherein excess foam is removed from the cavity through the second port in the second lumen, and
wherein the excess foam is discharged from the second lumen through the third port.

12. The foam delivery system of claim 11, wherein the valve is provided on the first lumen.

13. The foam delivery system of claim 10, wherein the pump system is a manual pump system.

14. The foam delivery system of claim 10, wherein the pump system is an electronically controlled foam delivery pump system.

15. The foam delivery system of claim 14, further comprising:
one or more processors configured to send signals to control foam delivery; and
an overflow detection sensor configured to detect overflow foam.

16. The foam delivery system of claim 15, wherein the overflow detection sensor is a camera.

17. The foam delivery system of claim 14, further comprising:
a disconnect,
wherein the disconnect is configured to removably couple a first portion of the lumen with a second portion of a lumen such that the first portion of the lumen.

18. The foam delivery system of claim 14, wherein the lumen is a dual lumen.

19. The foam delivery system of claim 18,
wherein the dual lumen includes a first lumen and a second lumen,
wherein the first lumen includes a first port through which foam is delivered to the cavity,
wherein the second lumen includes a second port and a third port,
wherein excess foam is removed from the cavity through the second port in the second lumen, and
wherein the excess foam is discharged from the second lumen through the third port.

20. The foam delivery system of claim 1, wherein the foam delivery system is coupled with an endoscopy system, a laparoscopy system, or a system used in veterinary applications.
